(19) United States
(12) Patent Application Publication
(10) Pub. No.: US 2023/0375492 A1
(43) Pub. Date: Nov. 23, 2023

(54) METHODS AND APPARATUS FOR SENSING VOLATILE ORGANIC COMPOUNDS AND GASES RELEASED FROM ELECTROCHEMICAL CELLS

(71) Applicant: Purdue Research Foundation, West Lafayette, IN (US)

(72) Inventors: Vilas Ganpat Pol, West Lafayette, IN (US); Palwinder Kaur, West Lafayette, IN (US)

(73) Assignee: Purdue Research Foundation, West Lafayette, IN (US)

(21) Appl. No.: 18/107,115

(22) Filed: Feb. 8, 2023

Related U.S. Application Data
(60) Provisional application No. 63/308,477, filed on Feb. 9, 2022.

(51) Int. Cl. G01N 27/16; G01R 31/392; G01N 27/07; G01R 31/385

(52) U.S. Cl. CPC G01N 27/16; G01R 31/392; G01N 27/07; G01R 31/385

(57) ABSTRACT

A battery management system (BMS) to detect conditions favorable to thermal runway. The BMS includes Interdigitated Platinum Electrode (IPE) on a substrate. The IPE contains a coating containing PEDOT:PSS (poly(3,4-ethylenedioxythiophene) polystyrene sulfonate). The system has an impedance measuring system (IMS) to measure impedance changes caused in the IPE due to exposure to volatile organic compounds (VOC's) resulting from decomposition of electrolyte in a battery. The impedance changes are indicative of release of the VOC's from the battery signaling a temperature increase in components of the battery. A method of inferring the concentration of VOC's produced by an electrochemical cell. The method includes providing a sensor integrated into the electrochemical cell, the sensor being IPE on a substrate containing a coating containing PEDOT:PSS, recording the impedance response of the sensor to the VOC's released by the electrochemical cell and inferring the concentration of the VOC's.

### CROSS-REFERENCE TO RELATED APPLICATIONS

The present U.S. patent application is related to and claims the priority benefit of U.S. Provisional Patent Application Ser. No. 63/308,477, filed Feb. 9, 2022, the contents of which are hereby incorporated by reference in their entirety into the present disclosure.

### TECHNICAL FIELD

The present disclosure generally relates to methods and apparatus for chemosensing or sensing the chemical compounds volatile organic compounds released from electrochemical cells, especially Li-ion batteries.

### BACKGROUND

This section introduces aspects that may help facilitate a better understanding of the disclosure. Accordingly, these statements are to be read in this light and are not to be understood as admissions about what is or is not prior art.

Lithium-ion batteries have revolutionized electrochemical energy storage The need for high power, energy dense batteries increases exponentially in importance as portable electronics and electrical vehicles further become integrated in our daily lives. Recent thermal runaway incidents however underscored the importance of reliability and safety. Several steps are involved in the thermal runaway process of electrochemical cells such as but not limited to Li-ion batteries. Catastrophic thermal runaway may be initiated through several mechanisms including overheating, overcharging, or short-circuit. At an elevated temperature of 80-120° C., the metastable solid electrolyte interphase (SEI) passivation layer exothermically decomposes, further raising battery temperature. This leads to continuous reactions between the unstable electrolyte and reductive lithium in the anode. Once the internal temperature reaches 160° C., the polypropylene separator melts and can no longer prevent contact of the electrodes. The resulting short circuit causes joule heating and eventually reaches the decomposition temperature of the metal oxide cathode at approximately 220° C. Generated oxygen causes a self-sustaining highly exothermic reaction that results in battery venting, explosion, and fire. Vented gases generated from electrolyte reactions may be flammable or toxic, such as carbon monoxide, methane, carbon dioxide, hydrogen, and hydrogen fluoride. Additionally, volatile organic compounds (VOCs) such as ethyl methyl carbonate (EMC), dimethyl carbonate (DMC), and methyl formate (MF) are released. Recent research indicates early detection of these gases coupled with a BMS capable of suppressing the thermal runaway event may enhance safety be preventing chain reactions in adjacent Li-ion cells. At temperature around 60° C., batteries emitting a few gases and VOCs. At this temperature, before the actual thermal runaway starts happening in the battery, by detecting certain gases, an accident can be avoided. Researchers demonstrated use of a MEMS MOS gas sensor for detection of VOCs leaking from the Li-ion Batteries. Various researchers also reported early detection of thermal runaway by sensing carbon dioxide, methane, propane, carbon monoxide and many other gases using metal oxide-based semiconductor sensors. However, these sensors require high temperatures (>200° C.) to function, making it unsuitable for real time monitoring of thermal runaways. On the other hand, Conducting Polymers are the materials widely used for gas sensing at room temperature conditions.

Conducting polymers are studied for applications in smart electronics, batteries, and displays because of their facile processing and tunable engineering properties (conductivity, flexibility, etc.). Recently they have also been identified as an attractive sensing material for impedimetric gas sensors due to semiconducting properties at room temperature. Conventional metal-oxide-based sensors require high temperatures (>200° C.) to function. Conducting polymers therefore is a scalable, cost effective, and energy efficient option for gas sensing at the cost of selectivity. Conducting polymers are often used for detection of multiple compounds simultaneously, specifically volatile organic compounds (VOCs). Conducting polymers investigated in the literature for sensing applications include polyaniline, polypyrrole, polythiophene. PEDOT:PSS (poly(3,4-ethylenedioxythiophene) polystyrene sulfonate) stands out among alternatives with its high thermal stability (up to 120° C.), adjustable conductivity (10⁻³-10³ S cm⁻¹), facile dispersion in water, and strong affinity for Volatile Compounds such as ethanol, ammonia, methanol, acetone, and formaldehyde. PEDOT:PSS conducts through conjugation of holes on the oxidized PEDOT backbone. Anionic PSS polymer stabilizes holes and forms a macromolecular salt with PEDOT. The presence of electron donating and receiving groups leads to sensitivity for many VOCs, and the degree of oxidation on PEDOT may be tuned to alter conductivity for optimized signal to noise ratio of chemosensors. Chemosensor is also refereed as chemoreceptor or molecular sensor, where sensory receptor responds to chemical substance when comes in contact. Various electrochemical methods are employed to capitalize upon changes in conductivity that result from coordination of active sites with VOCs. Techniques include voltammetry, amperometry, and electrochemical impedance spectroscopy (EIS). In contrast to other listed methods, EIS provides a multidimensional response by measuring changes in impedance with an applied AC perturbation over a finite frequency range. Multi-dimensionality results from simultaneous measurement of real and imaginary impedance, which enables further extraction of parameters including dielectric constant, charge transfer resistance, double layer capacitance, diffusion constant, etc. Information rich measurements are therefore exploited to detect multiple gases using a single sensing material.

Current capabilities include preventing overcharging of batteries via electronic circuitry that sits on the top of the battery electrodes. However, however that does not inform early to the user that battery is leading to thermal runaway. Hence there is an unmet need for early detection of potential thermal runaway of the battery inform early to the user that battery is leading to thermal runaway.

### BRIEF DESCRIPTION OF DRAWINGS

While some of the figures shown herein may have been generated from scaled drawings or from photographs that are scalable, it is understood that such relative scaling within a figure are by way of example, and are not to be construed as limiting.

FIG. 1 shows schematic representation of experimental setup depicting sensing of VOCs using PEDOT:PSS connected to impedimetric spectroscopy.

FIG. 2 shows SEM Image and EDS mapping of Electrode coated with PEDOT:PSS

FIG. 3 is Impedance response of the sensor without analyte for t minutes.

FIG. 4 shows Impedance vs Time response of PEDOT:PSS sensor for three analytes (15 PPM) at frequency of 100 KHz.

FIGS. 5A through 5I show Nyquist plots of impedance response of the sensor for three different concentrations of three VOCs (Ethanol, Ethyl-Methyl Carbonate and Methyl Formate)

FIG. 6 shows an equivalent electrical circuit with observed and fitted data for impedance response of the sensor in absence of analyte.

FIGS. 7A through 7F show clusters of different classes based on value of Equivalent Electrical Circuit parameters (Charge Transfer Resistance, Admittance and exponent)

FIGS. 8A through 8F show Principal Component Analysis of different analytes with respect to three different concentrations using (7A-7C) 25 datapoints for each analyte (7D-7F) using only 5 (t=0) datapoints for each analyte.

FIG. 9 shows 2D PCA for methyl Erick.

FIGS. 10 and 11 schematically depict configurations in which the VOC sensing element is placed inside the battery.

FIGS. 12 and 13 schematically depict configurations in which the VOC sensing element is placed inside a battery pack.

### SUMMARY

A battery management system (BMS) to detect conditions favorable to thermal runway of the battery is disclosed. The BMS includes Interdigitated Platinum Electrode (IPE) on a substrate. The IPE contains a coating containing PEDOT:PSS (poly(3,4-ethylenedioxythiophene) polystyrene sulfonate) The system also includes an impedance measuring system (IMS) capable of measuring impedance changes caused in the IPE due to exposure to volatile organic compounds and gases resulting from decomposition of electrolyte in the battery. The impedance changes are indicative of release of the volatile organic compounds and gases and mixtures thereof from a battery signaling a temperature increase in components of the battery which can cause a thermal runaway.

### DETAILED DESCRIPTION

For the purposes of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein being contemplated as would normally occur to one skilled in the art to which the disclosure relates.

This disclosure details fabrication and utilization of a system and method for Impedimetric Chemo sensing of Volatile Organic Compounds Released from Li-ion Batteries. Its applicability extends beyond the analytes and the electrochemical cells described here. In studies leading to this disclosure, A submicron (˜0.15 μm) thick poly(3,4-ethylenedioxythiophene) polystyrene sulfonate (PEDOT:PSS) sensor film is coated on a platinum electrode through a facile aqueous dispersion. The resulting sensor reliably detected different Volatile Organic Compounds (VOCs) released during the early stages of thermal runaway of lithium ion batteries (LIBs) even at low concentrations. The single-electrode sensor utilizes impedance spectroscopy to measure ethyl methyl carbonate and methyl formate concentrations at 5, 15, and 30 PPM independently and in various combinations using ethanol as a reference. In contrast to a DC resistance measurement which provides a single parameter, impedance spectroscopy provides a wealth of information, including impedance and phase angle at multiple frequencies as well as charge transfer resistance and constant phase elements in obtained Nyquist plots. Different analytes influence measurement of different parameters to varying degrees, enabling distinction using a single sensing material. The response time for ethyl methyl carbonate was measured 6 seconds. Principle component analysis method using first three Principal Components (PCs) holding more than 95% of data discriminates different classes of analytes efficiently. Application of low-power PEDOT:PSS based gas sensors will facilitate cost-effective early detection of VOCs and enable battery management system (BMS) to obtain early warning,

In this disclosure, as mentioned above, PEDOT:PSS conducting polymer is used as a gas sensing material for the detection of VOCs released during battery venting: ethyl methyl carbonate, and methyl formate. Ethanol is chosen as a standard to evaluate sensitivity for the VOC analytes, as the sensitivity of PEDOT:PSS for ethanol has been previously tested and characterized. Ethanol proves the sensitivity of material toward VOCs and explains the sensing phenomenon based on available literature. Although, Alcohols are also produced as the byproducts of chemical reactions of electrolytes during thermal runaway. Equivalent Electrical Circuit parameters were extracted from the EIS data by circuit fitting, to understand the sensor physics. Through principal component analysis of high-dimension EIS data having 61 frequencies as the features, 3 principal components were extracted which retain 95% of the variability of the original dataset and decouple different concentration responses to analytes. Use of multi-variable sensor coupled with Battery management system enhances the Battery safety by generating an early warning by detecting gases or VOCs released from the batteries or battery packs.

In experiments to leading to this disclosure, the following methods and materials were utilized: Dry, re-dispersible PEDOT:PSS pellets (1 S cm⁻¹) were purchased from Sigma Aldrich and used without additional purification. PEDOT:PSS was first dispersed in Millipore water to make a 1 wt. % solution. Next, ethanol (200 proof, purchased from KOPTEC, Decon Labs) was added, and the solution was stirred for 15 minutes under sonication to obtain a homogeneous 0.3 wt. % solution. Sensors were fabricated by spin-coating 10 μL of the PEDOT:PSS solution on an interdigitated platinum electrode (Purchased from Case Western University, OH) at 4000 rpm. This process was repeated once for a total of 20 μL of solution coated.

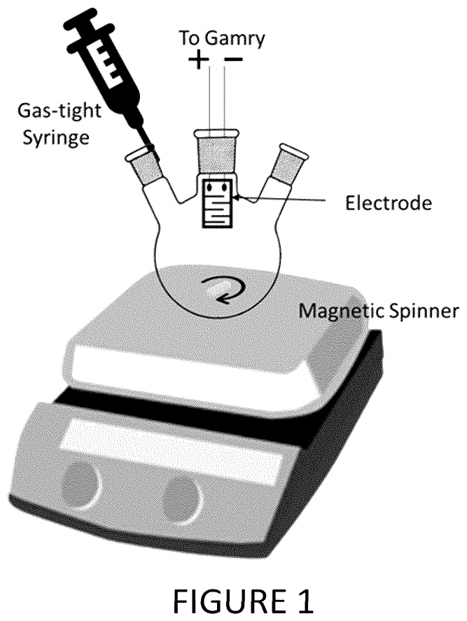

Three VOCs were chosen as analytes to test the sensor response, including ethanol, ethyl methyl carbonate (purchased from Sigma Aldrich with 99% purity) and methyl formate (purchased from Sigma Aldrich with 99% purity). The three analytes are tested at three concentrations of 5, 15, and 30 PPM. A Gamry Reference 600+ potentiostat measures impedance over a frequency range of 1 Hz to 1 MHz with an amplitude of 10 mV. Equivalent circuits are fit using Gamry Electrochemistry Analyst software. Each scan took approximately five minutes to cover the range of frequency. The response was observed for a total time duration of 25 minutes with 5 different readings at intervals of 5 minutes. Trials were repeated 5 times. Measurements were performed at room temperature in a custom made 25 mL chamber schematically shown in FIG. 1. The chamber was purged for 5 minutes with nitrogen after trial.

Vapor pressure of the VOC at temperature T (K) was calculated using Clausius Clapeyron Equation given by equation (1).

$$ln ( \frac{{P}_{2}}{{P}_{1}} ) = \frac{- Δ {H}_{vap}}{R} ( \frac{1}{{T}_{2}} - \frac{1}{{T}_{1}} ) \qquad ( 1 )$$

Where P is the vapour pressure (Kpa), ΔHᵥₐₚ is the enthalpy of vaporization (KJ/mol), R is the gas constant (J/K·mol), and T is the temperature (K).
   Concentration of the analyte was calculated using equations (2) and (3) [27].

$$volume of solvent evaporated = ( headspace volume ) * ( \frac{P°}{R T} ) * ( \frac{M}{ρ} ) \qquad ( 2 )$$

$$Concentration ( ppm ) = \frac{volume of solvent evaporated ( ul )}{total volume of the chamber ( ml )} \qquad ( 3 )$$

Where P° is the vapor pressure(kPa), R is the gas constant (J/K·mol), T is the Temperature (K), M is the molecular weight (g/mol), and ρ is the density (g/mL). The liquid sample was stored in an auto sampler vial for a day, and gas was collected from the headspace using a gas tight syringe (manufacturer, volume) and transferred into the testing chamber through a rubber septum. Principal Components were calculated using inbuilt library functions of Python.

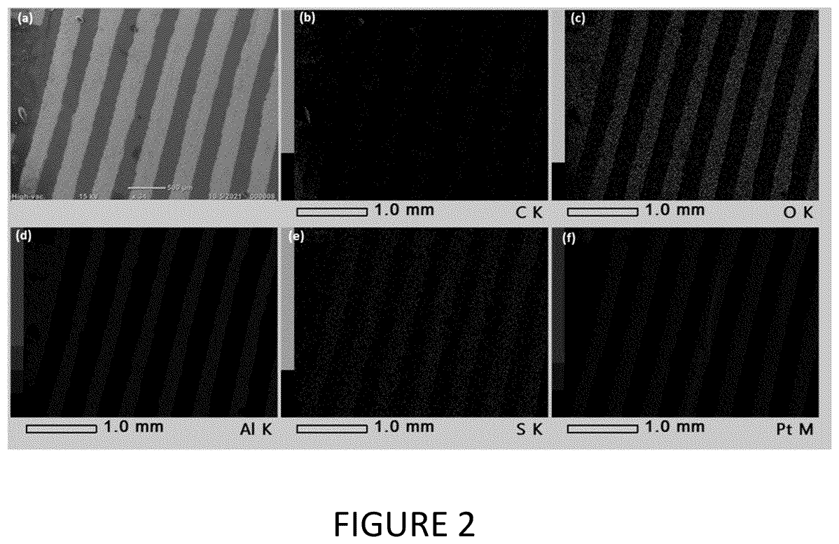

Conducting Polymer PEDOT:PSS acts as the sensing material, selected for its high thermal stability (>100° C.) and tunable conductivity. Conductivity of the sensor is increased by using double layer spin coating. FIG. 2 shows SEM/EDS images for the prepared interdigitated electrodes. For both the bare and coated (FIG. 2(section a)) electrodes, dark areas are the non-conductive alumina substrate, and the lighter areas are the Pt branches of the interdigitated electrode. EDS atomic mapping FIG. 2(sections c,d,f)) shows that the light areas are highlighted with Pt, and the darker areas with O and Al. Atomic mapping also shows that with the PEDOT:PSS coating, the quantity of S atoms increases due to the sulfone groups from ND(Not-Detectable) to 0.04%, and C from 15.52% to 29.24%. The S and C elements appear to evenly coat the electrode, showing the efficacy of spin-coating in achieving a uniform sensing surface. The small change in atomic percent may be due to the small thickness (0.15 μm) of the coated layer.

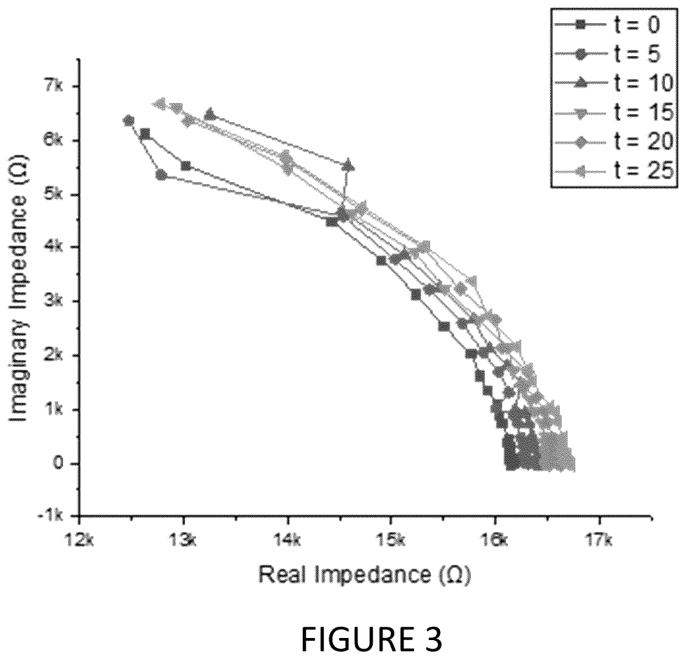

Experiments were performed at room temperature, and data represented as Nyquist plots, where in-phase (real) and out-of-phase (imaginary) components of impedance are represented on the axes. Frequency implicitly decreases from left to right, and is collected over a range of 1 Hz to 1 MHz. FIG. 3 shows the baseline response of the sensor after purging the chamber with nitrogen. Changes between measurements at different time intervals are likely due to the high sensitivity of PEDOT:PSS to humidity. Purging the chamber with nitrogen gas decreases the humidity up to less than 10%. After removing nitrogen inlet from chamber opening, the humidity might tend to restore as per environment humidity. The change observed in the baseline of the impedance response is due to the increased humidity in the chamber. Although the change is observed as constant between two consecutive readings at different time interval. Moisture potentially interacts with the sensor as air from the environment enters the chamber, while closing the nitrogen inlet. In the case of analyte injection, the change in impedance after baseline is significant and in proportion to the concentration of the analyte.

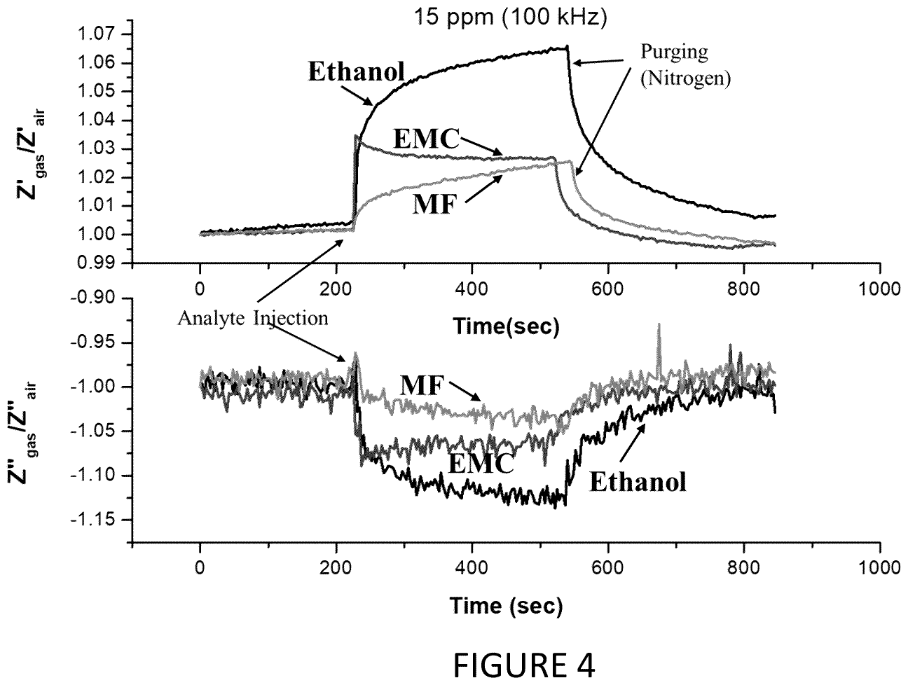

FIG. 4 shows the Impedance (normalized real and imaginary) vs time (seconds) response of the PEDOT:PSS sensor for three different analytes ethanol, EMC and MF (15 PPM each) at frequency of 100 KHz. The response time (time required to reach 90% of saturated value) observed for Ethanol, MF is approximately 234 seconds, 159 seconds and 6 seconds, respectively. For EMC, sensor shows an abrupt response within few seconds. The sensor has similar recovery period for all three analytes (250 seconds approximately).

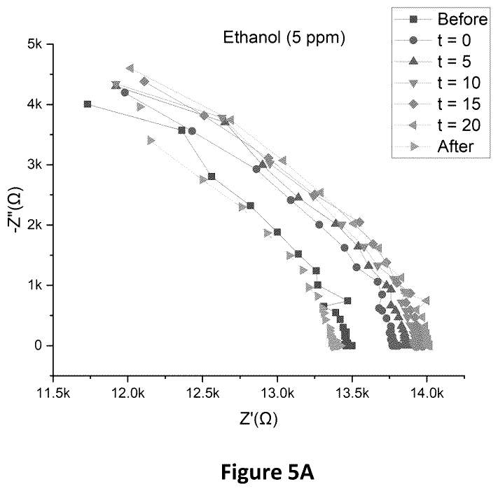

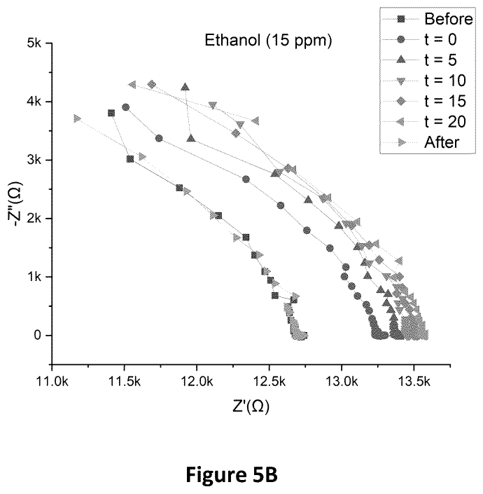

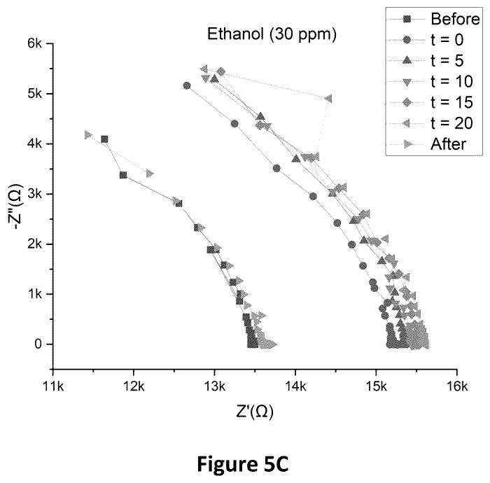

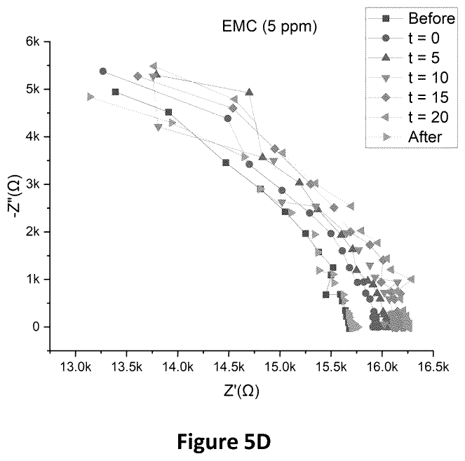

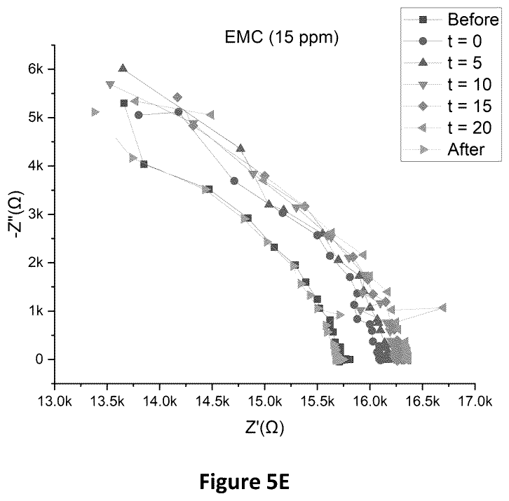

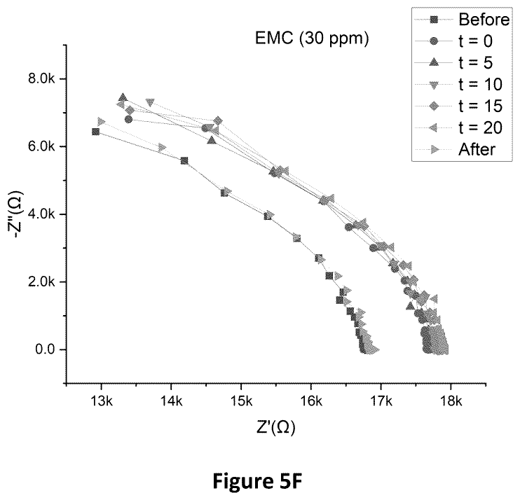

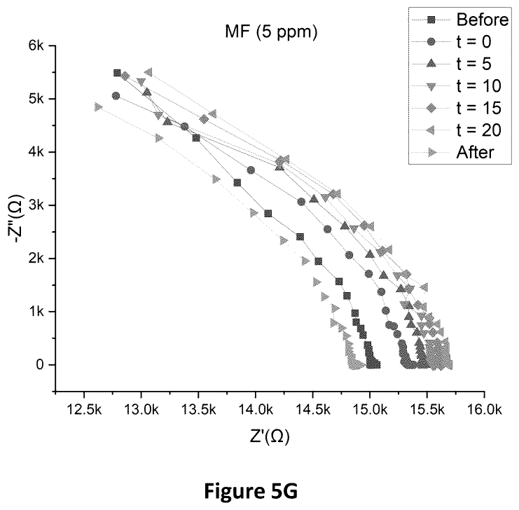

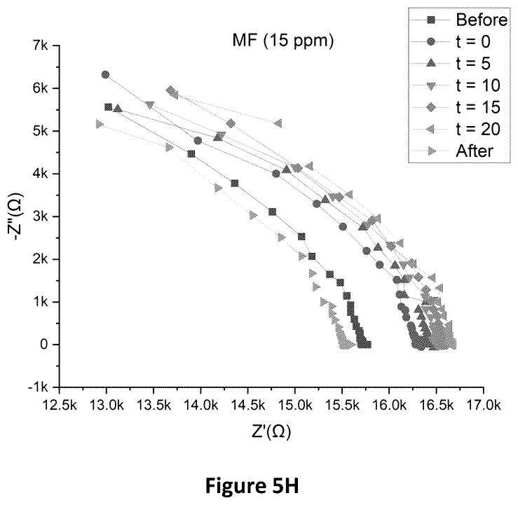

FIGS. 5A through 5H show the altered impedance response over a span of 25 minutes after addition of a single VOC analyte (ethanol, EMC, and MF), at concentrations of 5, 15, and 30 PPM for each. Even in the first measurement 5 minutes after injection of the analytes, impedance measurably increases, indicating excellent sensitivity. Over the course of 25 minutes, the impedance continues to gradually increase as additional analyte diffuses into the polymer film and adsorbs to active sites, hindering the conjugation of holes through the PEDOT backbone. As the sensor becomes saturated over the time intervals, the response become approximately constant. After purging with Nitrogen for 5 minutes at 20 PSI, the sensor reliably returns to the baseline as analyte desorbs. The absence of permanent changes to the baseline highlights the chemical resiliency of the conducting polymer to reactions with the analyte at room temperature. Trials were repeated for a total of 5 times each, suggesting reliability and longevity of the PEDOT:PSS sensor. Overall, the changes in delta response and reversibility demonstrate effectiveness of PEDOT:PSS as a sensing material for the VOCs of interest.

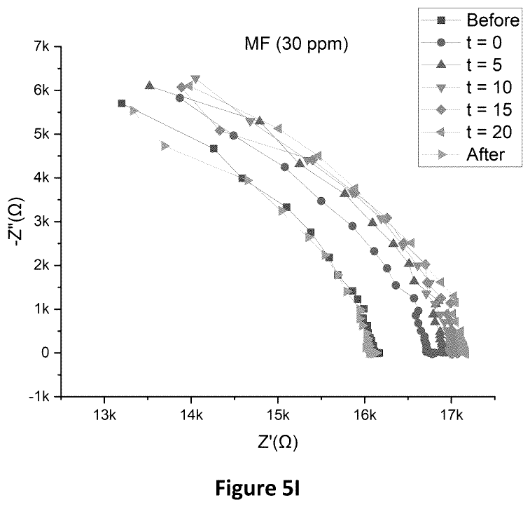

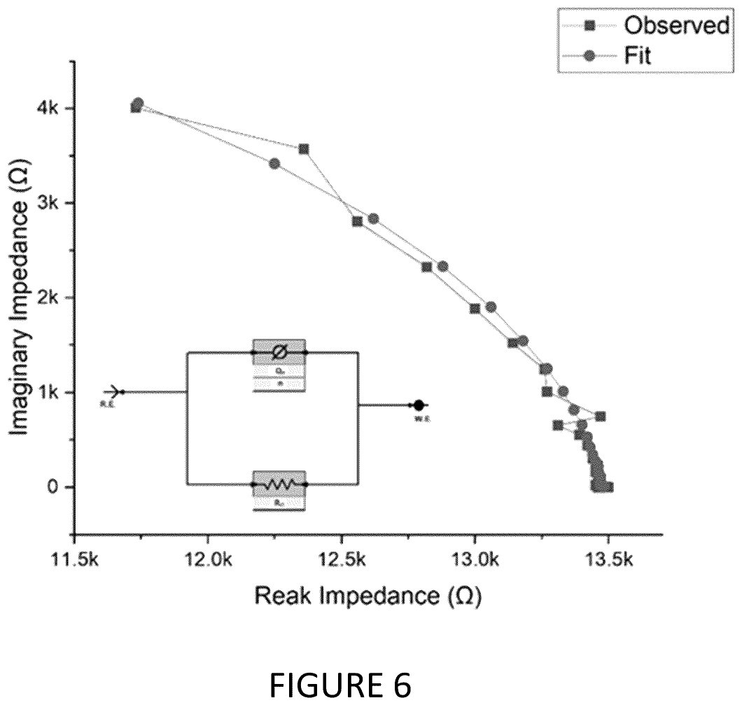

Through visual inspection of FIGS. 5A through 5I, one can qualitatively observe that changes (increase) to the real impedance are more pronounced at lower frequencies as concentration varies. In contrast, the changes to the imaginary impedance are more significant at higher frequencies, and magnitude of the delta response depends greatly upon the analyte and its concentration as well. To aid in the quantification of responses and obtain additional information through extraction of metavariables, a model equivalent circuit was fit to the experimental data, as shown in FIG. 6. Elements of the equivalent circuit are selected based on the observed data and intuition about the physical components of the sensor. A parallel combination of a resistor and constant phase element (CPE) provides an excellent fit to the Nyquist plot in FIG. 6, giving a goodness-of-fit value of <10⁻⁵. We obtain similar convergence in all fits, demonstrating this choice of equivalent circuit accurately represents the physical circuit. The equation for the equivalent circuit is given in Equation 4,

$${Z}_{R ‖ C} = {Z}_{R e} + j {Z}_{i m} = \frac{R}{1 + {R}^{2} {Q}^{2} {ω}^{2}} - \frac{j ω Q}{1 + {R}^{2} {Q}^{2} {ω}^{2}} \qquad ( 4 )$$

where Z is impedance and ω is the angular frequency. Here, charge transfer resistance (R_{CT}) represents electron impedance with the small applied potential perturbation of 10 mV [30], and is given by Equation 5,

$${R}_{ct} = \frac{R T}{η F {i}_{0}} \qquad ( 5 )$$

where R is the universal gas constant, T the temperature, η the activation overpotential, and i₀ the exchange current density (A/m²). Exchange current density represents the intrinsic rates of electron transfer between the electrode and an analyte. Therefore, directly influencing the charge transfer resistance. Amplitude of applied perturbation is kept small to obtain a pseudolinear behavior of the electrochemical sensor. Capacitive charge storage from the electric double layer is best represented through a CPE (Q) as shown in Equation 6 [33].

$${Z}_{CPE} = \frac{1}{{Y}_{CPE}} = \frac{1}{{{Q}_{0} ( j ω )}^{n}} \qquad ( 6 )$$

Here, Q₀ is the capacitance and n is a value between 0 and 1, where 1 represents an ideal capacitor and 0 represents the pure resistor. We observe a value for n of approximately 0.95, indicating pseudocapacitive behavior of the electric double layer. Parameters obtained from fits to experimental data encompass physical phenomena that vary in importance for different analytes. This is useful for distilling critical variables of the information rich EIS data for quantification of VOC mixtures using a single sensing material. The sensor seems to be more responsive to ethanol as compare to other analytes. Such phenomenon can be explained by the presence of weak Vander wall forces in ethanol molecule and low molecular weight. As the phenomenon of the adsorption depends upon the mass and nature of the adsorbate. EMC and MF have comparatively high molecular weight and presence of double bond. The other factor like temperature and pressure were kept constant.

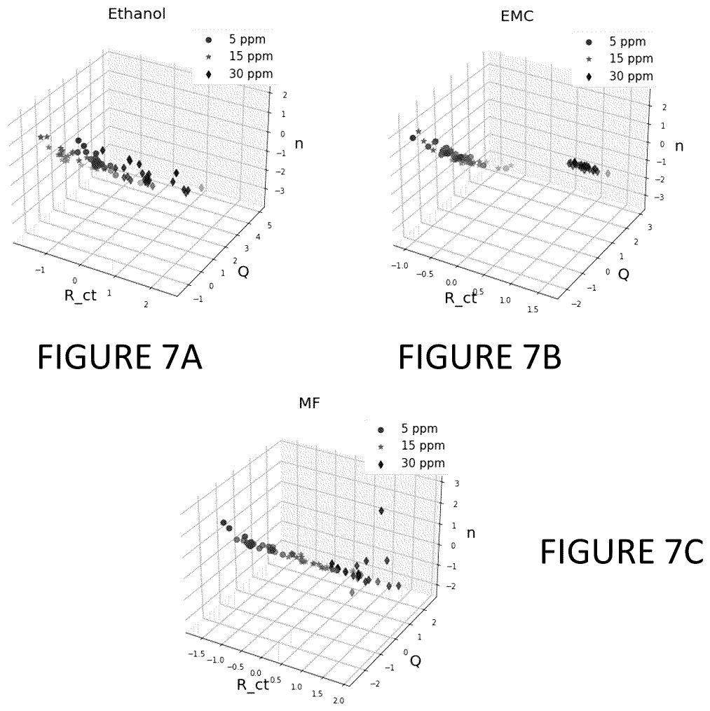

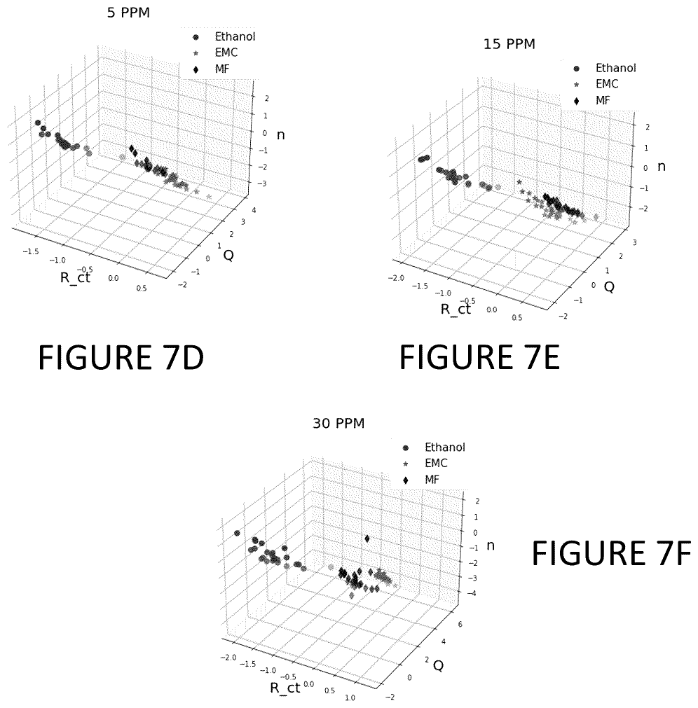

FIGS. 7A through 7F show three-dimensional plots of sensor response with fitted variables R_{CT}, Q₀, and n represented on the axes. Values are normalized to univariate mean. A total of 25 data points are considered for each class as trials are recorded 5 times at intervals of 5 minutes (total 25 mins), and repeated 5 times. FIGS. 5A through 5I reveal that the changes between time intervals are negligible as compared to the impedance change at t=0, and therefore are represented together on the plot. Charge transfer resistance plays a major role in discriminating between concentrations of a single analyte (FIGS. 7A-7C)), as well as different analytes at the same concentration (FIGS. 7D-7F). The charge transfer resistance proves to be a critical parameter, as we observe distinct changes in charge transfer resistance values for most classes. For example, in FIG. 7A, increasing concentration of ethanol causes a corresponding increase in the R_{CT}, demonstrating potential for accurate sensing. For EMC however (FIG. 7B), little change is observed between 5 and 15 PPM, foreshadowing difficulty in quantifying EMC at low concentrations if relying only upon the charge transfer resistance. Multidimensional information obtained from EIS thankfully aids in discerning between these classes. FIGS. 7D-7F show that EMC (red) and MF (blue) are better differentiated through changes in Q₀ and n, at the same concentration. Despite having similar R_{CT} values, these classes can therefore be distinguished. In FIG. 7F, admittance plays a particularly critical role in differentiating between EMC and MF at 30 PPM. At high concentrations, extensive coordination of the analytes alters the dielectric constant of the material, indirectly altering the double layer capacitance to varying degrees for different analytes. The value of exponent is less than 1 indicating the properties of impure capacitance. The value of the exponent (<1) further decreases with increases in concentration, causing greater pseudocapacitive behavior and further deviation from ideal capacitance.

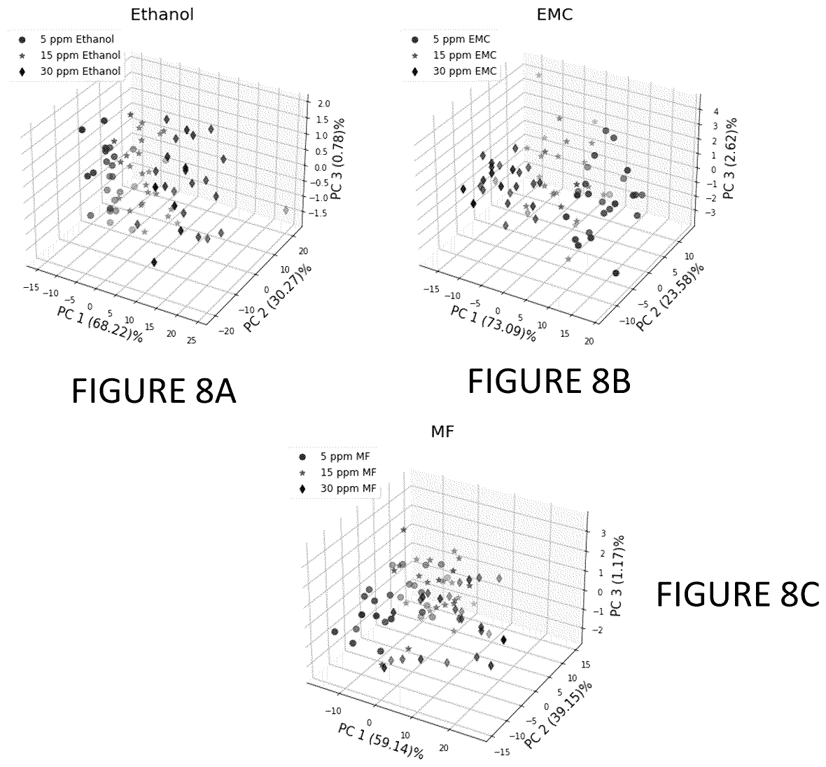

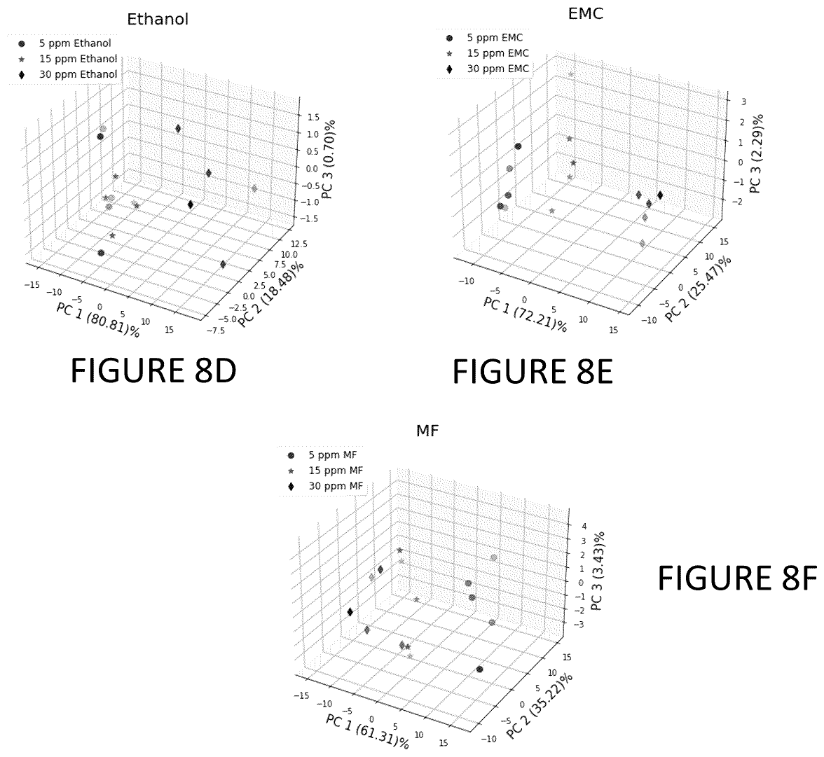

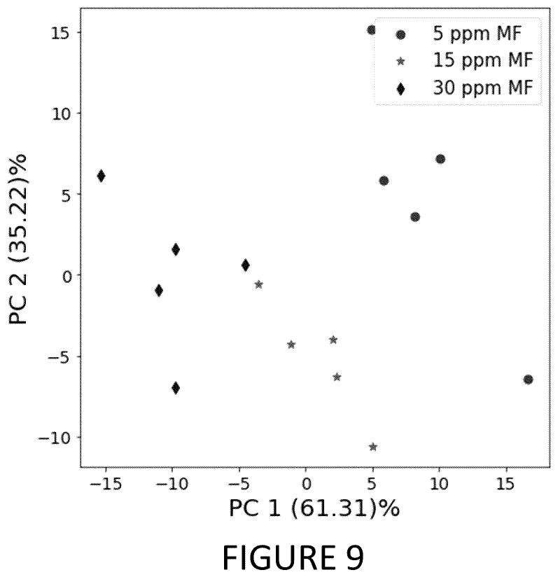

As was observed in FIGS. 7A-7F, differences between classes may be clearly seen in changes to parameters. However, due to the large size of the dataset and subtlety in some observed changes, it is necessary to automate the selection of critical parameters that facilitate distinction of the different classes. Towards this end we apply principal component analysis (PCA), a method of dimension reduction for large datasets. The method reduces dimensionality to an optimal extent that reduces complexity while preserving variability and enables effective visualization, parameter selection, and computation. The dataset with reduced dimensions includes new variables denoted principal components (PCs), representing the eigen vectors computed from correlation matrix of the complete dataset. EIS data is collected at 61 different frequencies between 1 MHz to 1 Hz, and at each frequency both real and imaginary impedance are measured, giving a total number of 122 independent variables/features. PCA transforms such large data sets in selected number of PCs, which still enable classification of the different analytes and concentrations. Three PCs explaining more than 95% of the dataset were distilled and used as axes in three-dimensional PCA plots for experimental data of varying analyte and concentration (FIGS. 8A-8F). The feature value is the change in the impedance of the sensor in the presence of analyte with respect to the impedance of the sensor in the absence of analyte. FIGS. 8A-8C again show 25 data points for each sample from data collection at multiple time intervals and repeated trials. Other than frequency, time is a variable that can alter the response of the sensor to analyte. It is important to compare the sensor response in a fixed time window. FIGS. 8D-8F therefore, only represent the first-time interval reading for each sample. From these PCA plots we see clear discrimination of the data points between samples of different concentrations and analyte into their respective classes, with a single exception for an outlier for EMC at 5 PPM (FIG. 8E). In FIG. 8F, lower concentrations of MF appear difficult to distinguish. However, visualization in a two-dimensional graph in FIG. 9 using only PC1 and PC2 reveals that these classes are actually clearly discriminated.

It can therefore be concluded that synergy of PCA and information-rich EIS enables accurate classification. Using frequencies as features with both real and imaginary impedance as the independent variable value increases the sensitivity and selectivity of the sensor.

Thus, sensors based on commercially available conducting PEDOT:PSS polymer offer a promising low-cost alternative to current metal-oxide based due to facile processing and low power requirements. In this study, we demonstrated great sensitivity for even low concentrations of VOCs which are released from venting batteries in the initial stages of thermal runaway. Sensitivity to three analytes, Ethanol, EMC, and MF is made possible through electrochemical impedance spectroscopy, which evaluates impedance at varying frequencies. Response varies at different frequencies for different analytes, enabling distinction and quantification using a single sensing material. Principal component analysis further demonstrates enhanced classification and reduces the number of parameters for efficient computation. With the application of machine learning algorithms, an efficient multivariable impedance-based gas sensor can be developed and integrated into a battery management system to generate early warning of thermal runaway and take preventative measures. The reported single sensor efficiently discriminates different concentrations of the three analytes, which is not be possible using a single DC resistance-based sensor. Future avenues towards enhancing the selectivity of the sensor involve optimization of different combinations of features. Such sensors have important implications towards enhancing personal safety as electric vehicles and small to mid-scale LIB energy storage become more widespread.

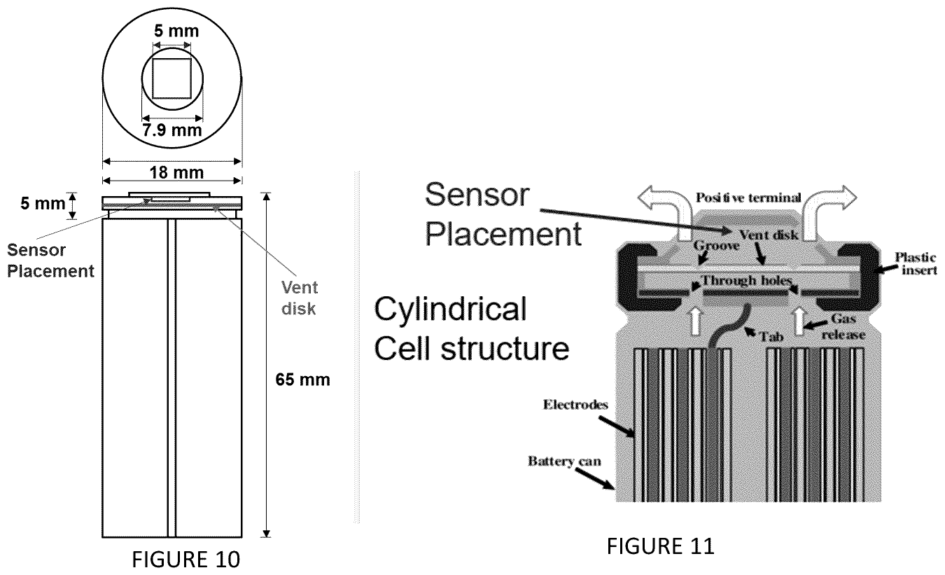

An important aspect to be considered is the location of the sensor described above. FIGS. 10 and 11 describe a location scheme where the VOC sensor is located inside the battery i.e. the electrochemical cell. This location ensures that the sensor is safe from the electrolyte or electrolyte vapor during the normal operation, and the vent disk grooves break with generation of gases. In this configuration, the sensor can generate early warning. Further, the sensor is safe from humidity and gases present in the atmospheric air.

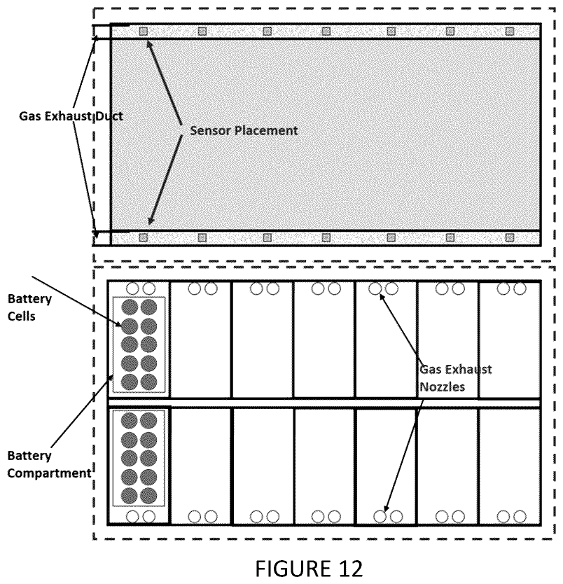

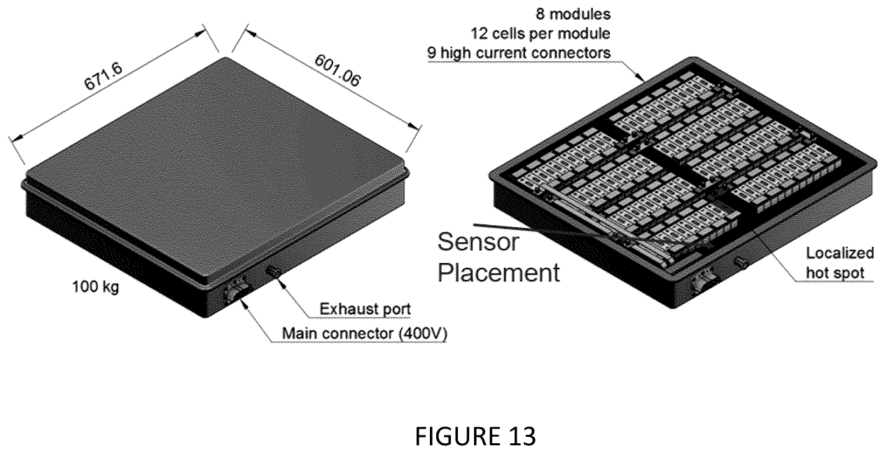

FIGS. 12 and 13 depict a location scheme wherein the sensor is located in the battery pack. This scheme allows continuous monitoring of battery environment, during charging, discharging or ideal. Early warnings can be negated by specified changes in sensor response as a function of increased resistance in the battery system. This can also allow detection of faulty cells, namely cells opened because of mechanical or vibrational impacts. The sensor could work in humidity and gases present in atmospheric air with decreased sensitivity. As shown in FIG. 13, placing the sensor near to exhaust port of large battery pack comprising thousands of smaller cylindrical or prismatic cells. allows the above-mentioned advantages.

In some experiments leading o this disclosure, the single sensor was implemented to detect nine different binary mixtures and five different ternary mixtures chosen randomly of three VOCs, namely ethyl methyl carbonate, methyl formate, and ethanol with 5, 15, and 30 ppm concentrations. Equivalent electrical parameters like charge transfer resistance and constant phase elements fitted with the goodness of fit value less than 10⁻⁵ and the Principal Components Analysis (PCA) method was used to distinguish response into different classes.

In additional experiments, the sensor response was monitored at elevated (40° C., 55° C. and 70° C.) temperatures for single analytes and binary mixtures of two VOCs with 5 ppm, 15 ppm and 30 ppm concentrations. Equivalent electrical parameters are derived from impedance data, the sensor response was computed by Principal Component analysis at different temperatures and support vector machine learning algorithm achieved almost 100% classification accuracy.

Based on the above description, it is an objective of this disclosure to describe a battery management system (BMS) to detect conditions favorable to thermal runway of the battery. The BMS includes, an Interdigitated Platinum Electrode (IPE) on a substrate, wherein the IPE contains a coating comprising PEDOT:PSS and an impedance measuring system (IMS) capable of measuring impedance changes caused in the IPE due to exposure to volatile organic compounds and gases resulting from decomposition of electrolyte in the battery. It should be noted that the impedance changes are indicative of release of the volatile organic compounds and gases and mixtures thereof from a battery signaling a temperature increase in components of the battery which can cause a thermal runaway. In the battery management system of claim 1, the IPE can be located inside or outside the battery and the IMS is located outside the battery. In some embodiments of the BMS of this disclosure, the IPE has a surface to volume ratio in the range of 700 (mm⁻¹) to 2100 (mm⁻¹). It should be recognized that higher surface area to volume ratios increase the number of sites for sensor analyte interaction with the IPE, thereby increasing the sensor sensitivity. In some embodiments the ratio surface to area to volume as measured ranged from 706 (mm)²/1 (mm)³ to 2076 (mm)²/1 (mm)³. In some embodiments of the BMS of this disclosure, the IPE has a surface roughness in the range of 0.5 micrometers to 1.5 micrometers. In one particular embodiment the measured average surface roughness of the IPE was 718.445 nm with root mean square height of 1458.138 nm. Increased surface roughness increases the surface area to volume ratio having the same effect on sensor sensitivity as explained above.

In some embodiments of the BMS of this disclosure, the substrate is a refractory oxide. Refractory oxides suitable for this purpose include, but not limited to, alumina and silica. In some embodiments of the BMS of this disclosure, the volatile organic compounds can be one or more of a group comprising, but not limited to ethanol, ethyl methyl carbonate, methyl formate individually or their mixtures. Ethylene, dimethyl carbonate, dimethyl ether, sulfur containing molecules and gases such as hydrofluoride, carbon monoxide, carbon dioxide, sulfides, fluorocarbons etc. can be detected with such methodology. In some embodiments of the BMS of this disclosure, the gases and VOCs mentioned above once detected by increase in resistance or impedance in the battery system, will be informed to BMS mitigating or lowering the chances of large catastrophic failure of battery by shutting down the current extraction or injection (discharging or charging) of the device. In some embodiments of the BMS of this disclosure, the impedance changes are in the range of 15 KΩ to 20 KΩ In some embodiments of the BMS of this disclosure, the concentration of any one of the VOC's and gases is in the range of 1 ppm to 50 ppm in a vicinity of IPE i.e. from 1 cm to 500 meters range. In some embodiments of the BMS of this disclosure the impedance change is measured within 5-10 seconds of exposure to at least one of the VOC's and gases. In some embodiments of the BMS of this disclosure, the impedance changes are measured in the frequency range 1 Hz-1 MHz. In some embodiments of the BMS of this disclosure, the dimensions of the IPE are 5 mm×5 mm×2 mm. It should be recognized that the size, shape and location of the developed chemosensor will be application-dependent. For example, in the cylindrical 18650 cell (18 mm diameter, 65 mm height and 0 means cylindrical) or analogous cylindrical with different diameter and height for enhanced energy density could utilize such sensor at the positive terminal above the pressure release valve. For the storage of Li-ion battery packs or battery fabrication facilities including dry rooms such sensor can be placed on the walls or ceiling and could be larger size (1 cm×1 cm×1 cm) or thinner or thicker. In some embodiments of the BMS of this disclosure, the spacing between the conductor (current collector, made from highly conducting and air stable conducting copper, platinum or gold) of the IPE is in the range of 100 nm to 1 mm. In some embodiments of the BMS of this disclosure, the BMS includes a microcontroller. A typical microcontroller (computer) possesses integrated complex or simple circuit planned to oversee a specific operation that is added to the battery system. The major components of the microcontroller are a processor, storage memory and input/output (I/O) connections, possibly on a single chip. Microcontrollers can be used to automatically control devices including but not limited to toys, power tools, remote controls, implantable medical devices, engine control systems and in this case Li-ion batteries and their health. In some embodiments of the BMS of claim 1, the IPE can sense and the IMS records the frequency range between 1 MHz to 1 Hz for single, binary and ternary mixtures of VOCs in the concentration range of 1 ppm to 100 ppm. It should be recognized that due to the materials chosen, and the locations schemes describe, some embodiments of the BMS of this disclosure can operate in a temperature range ranging from subzero temperatures such as to 110° C.

It is also an objective of this disclosure to describe a method of inferring the concentration of volatile organic compounds (VOC's) produced by an electrochemical cell in operation. The method includes providing a sensor integrated into the electrochemical cell or a plurality of cells, the sensor being Interdigitated Platinum Electrode (IPE) on a substrate, wherein the IPE contains a coating comprising PEDOT:PSS, and recording the impedance response of the sensor to the binary and ternary mixtures released by the electrochemical cell. In one experiment, the impedance is recorded using Gamry 600+ potentiostat in the frequency range of 1 Hz to 1 MHz with 10 mV Vᵣₘₛ at OCP (open circuit potential); and inferring the concentration of the volatile gases by the methods described in this disclosure which includes. principal component analysis applicable to the impedance measuring system.

While the present disclosure has been described with reference to certain embodiments, it will be apparent to those of ordinary skill in the art that other embodiments and implementations are possible that are within the scope of the present disclosure without departing from the spirit and scope of the present disclosure. Thus, the implementations should not be limited to the particular limitations described. Other implementations may be possible. It is therefore intended that the foregoing detailed description be regarded as illustrative rather than limiting.

Several references have been indicated parenthetically indicated numerals throughout the specification. These references are listed below. These references are also useful in understanding and enabling this disclosure. The contents of these references listed below are incorporated herein reference in their entirety into this specification.

1. A battery management system (BMS) to detect conditions favorable to thermal runway of the battery, the system comprising:
Interdigitated Platinum Electrode (IPE) on a substrate, wherein the IPE contains a coating comprising PEDOT:PSS;
An impedance measuring system (IMS) capable of measuring impedance changes caused in the IPE due to exposure to volatile organic compounds and gases resulting from decomposition of electrolyte in the battery,
wherein the impedance changes are indicative of release of the volatile organic compounds and gases and mixtures thereof from a battery signaling a temperature increase in components of the battery which can cause a thermal runaway.

2. The battery management system of claim 1, wherein the IPE is located inside or outside the battery and the IMS is located outside the battery.

3. The BMS of claim 1, wherein the IPE has a surface to volume ratio in the range of 700 (mm⁻¹) to 2100 (mm⁻¹).

4. The BMS of claim 1, wherein the IPE has a surface roughness in the range of 0.5 micrometers to 1.5 micrometers.

5. The BMS of claim 1, wherein the substrate is a refractory oxide.

6. The BMS of claim 4, wherein the refractory oxide is alumina or and silica.

7. The BMS of claim 1, wherein the volatile organic compounds comprise one or more of ethanol, ethyl methyl carbonate, methyl formate individually or their mixtures. Ethylene, dimethyl carbonate, dimethyl ether, and sulfur containing molecules

8. The BMS of claim 1, wherein the gases comprise one or more of hydrofluoride, carbon monoxide, carbon dioxide, sulfides, fluorocarbons or their mixtures

9. The BMS of claim 1, wherein the impedance changes are in the range of 15 KΩ to 20 KΩ

10. The BMS of claim 1, wherein the concentration of any one of the VOC's and gases is in the range of 1 ppm to 50 ppm ppm in a vicinity (1 cm to 500 meters) of IPE

11. The BMS of claim 1, wherein the impedance change is measured within 5-10 seconds of exposure to at least one of the VOC's and gases.

12. The BMS of claim 1, wherein the impedance changes are measured in the frequency range 1 Hz-1 MHz

13. The BMS of claim 1, where in the dimensions of the IPE are 5 mm×5 mm 5 mm×5 mm×2 mm

14. The BMS of claim 1, wherein the spacing between the conductor traces (made from highly conducting and air stable conducting copper, platinum or gold) of the IPE is in the range of 100 nm to 1 mm.

15. The BMS of claim 1, further comprising a microcontroller.

16. The BMS of claim 1, wherein the IMS records the frequency range between 1 MHz to 1 Hz for single, binary and ternary mixtures of VOCs in the concentration range of 1 ppm to 100 ppm.

17. The BMS of claim 1, wherein the IPE and the IMS are used in the temperature range of subzero (<0° C.) to 110° C.

18. The BMS of claim 1, wherein the battery is a lithium-ion battery.

19. A method of inferring the concentration of volatile organic compounds (VOC's) produced by an electrochemical cell in operation, the method comprising:
providing a sensor integrated into the electrochemical cell or a plurality electrochemical cells, the sensor being Interdigitated Platinum Electrode (IPE) on a substrate, wherein the IPE contains a coating comprising PEDOT:PSS;
recording the impedance response of the sensor to the binary and ternary mixtures released by the electrochemical cell by using an impedance measurement system; and
inferring the concentration of the volatile gases by the methods utilizing principal component analysis applicable to the Impedance measurement system.
